SEQUENCE LISTING (1 sequence)

SEQ ID NO: 1 - protein, 5 aa, Artificial Sequence
  RYIRS peptide tag
RYIRS

(19) United States
(12) Patent Application Publication
(10) Pub. No.: US 2012/0003667 A1
(43) Pub. Date: Jan. 5, 2012

(54) ELECTROPHORETIC TAG-BASED IN VITRO ASSAY TO QUANTIFY DIMERIZATION OF P66 AND P51 SUB-UNITS OF HIV-1 REVERSE TRANSCRIPTASE (RT)

(76) Inventors: Soumi Gupta, San Francisco, CA (US); Rajiv Dua, Manteca, CA (US); Douglas McCann, Cupertino, CA (US)

(21) Appl. No.: 12/664,634

(22) PCT Filed: Jun. 16, 2008
(86) PCT No.: PCT/US2008/007474

§ 371 Date: Oct. 25, 2010

Related U.S. Application Data
(60) Provisional application No. 60/944,069, filed on Jun. 14, 2007.

(51) Int. Cl. G01N 33/566

(52) U.S. Cl. 435/7.6; 435/7.1

(57) ABSTRACT

Methods for measuring hetero- and homodimerization of HIV reverse transcriptase (HIV RT) are provided using pairs of tagged probes and cleaving probes. The methods can be used, for example, to screen for modulators of HIV RT dimerization. Also provided are methods of determining whether a compound modulates HIV RT. Further provided are methods of determining whether a compound modulates formation of a complex between a p66 and p51, p66 and p66, or p51 and p55 subunits polypeptides of HIV RT.

### FIELD OF INVENTION

Provided herein are methods for measuring oligomerization of molecules, particularly dimerization of reverse transcriptase (RT).

### BACKGROUND OF THE INVENTION

Reverse transcriptase (RT) of the human immunodeficiency virus type 1 (HIV-1) plays a key role in the replication of HIV. It catalyzes the conversion of single-stranded genomic RNA into cDNA. The biologically relevant and active form of HIV-1 RT is a heterodimer containing two polypeptides, p66 and p51. The structure of HIV-1 RT has been elucidated by x-ray crystallography, and shows that p66 can be divided structurally into the polymerase and RNase H domains, with the polymerase domain further divided into the fingers, palm, thumb and connections subdomains. Although p51 has the same polymerase domains as p66, the relative orientations of these individual domains differ markedly, resulting in p51 assuming a closed structure.

The p51 polypeptide is derived from the p66 polypeptide by proteolytic cleavage of its C-terminal domain during viral maturation. The two subunits of 66 and 51 kDa are present in a 1 to 1 ratio. Structural analysis reveals three major contacts between p66 and p51, with most of the interaction surfaces being largely hydrophobic. The three contacts includes the fingers subdomain of p51 with the palm of p66, the connection subdomains of both subunits, and the thumb subdomain of p51 with the RNase H domain of p66. Several single amino acid substitutions in HIV-1 RT have been shown to inhibit heterodimer association. These include the mutations W401 A, L234A, G231 A, W229A, L289K, and others. L234A, G231A and W229A are located in the primer grip region of the p66 subunit and L289K in the thumb subdomain, and are not located at the dimer interface and probably mediate their effects indirectly through conformational changes in the p66 subunit.

The DNA polymerase and RNase H activities of HIV-1 RT are dependent on the dimeric structure of the enzyme. Because dimerization of these subunits is required for enzymatic activity, interference with the dimerization of HIV-1 RT could constitute a target for the development of anti-HIV compounds. Compounds that interfere with the formation and/or stability of the RT dimer may therefore represent a novel class of antiviral compounds.

Several publications disclose association-dissociation assays for measuring the kinetics of the p51-p66 dimerization process (Cabodevilla et al., 2001, Eur. J. Biochem. 2681163-172 and Morris et al., 1999, J. Biol. Chem. 274(35), 2491-24946). These assays measure the dimerization by size-exclusion HPLC, measuring the RNA-dependent DNA polymerase activity of the sample, immunoprecipitation, or by monitoring intrinsic fluorescence emission of the protein. Tachedjian et al. (2000 Proc. Nat. Acad. Sci. 97(12) 6334-6339) disclose a yeast 2-hybrid system to study the association-dissociation process of RT dimerization.

The known assays do not disclose a binding assay amenable for high throughput screening. Further, these methods are not convenient, sensitive, or cost effective. Development of a rapid, high-throughput, quantitative in vitro assay for RT dimerization would facilitate the identification of potential inhibitors of hetero- and homodimerization, could be useful as a diagnostic, could be used for understanding of the effect of connection domain mutants on RT dimerization, and the like. Thus, a need exists for methods for assaying for the dimerization of p51 and p66 polypeptides of HIV RT.

### SUMMARY OF THE INVENTION

Provided herein are high-throughput assays suitable, for example, for measuring dimerization of p66 and p51 subunits of HIV RT. The assays are useful, for example, for assessing large numbers of compounds for their activity on dimerization, e.g., heterodimerization or homodimerization. The general principle of the assay involves detecting cleaved fluorescent reporter tags, e.g., eTags.

In one aspect, p66 and p51 can be differently labeled. For example, wild type or mutant HIV-RT p66 or p51 coding sequences can be transferred into the pBAD/FLAG or pBAD/His expression vectors. Bacterially expressed, purified RT subunits can be mixed in equimolar ratios. The His-tagged subunit can be immobilized on a His-binding plate and then eTag-conjugated anti-FLAG antibody can be added to the heterodimer complex. The unreacted subunits and reagents can be washed, and the eTags bound to the heterodimer complex released and quantitated. Dimer formation can be quantified using an electrophoretic tag-based assay.

In another aspect, methods are provided that are suitable for measuring heterodimerization or homodimerization of HIV RT subunits. For example, samples can be incubated with a mixture of p66-FLAG/p51-His specific antibodies conjugated either with cleavable fluorescent reporter tags (eTags), or biotin, which binds a reporter tag-releasing agent (chemical scissor). After washing to remove unbound material, the amount of affinity-associated material can be assessed by measuring the level of a reporter moiety (eTags). Reporter molecules can be released based on proximity to the scissor in a photochemical reaction and quantitated following capillary electrophoresis. Such an assay can be used to measure homodimer or heterodimer formation by selecting antibodies that specifically bind the appropriate subunit.

Accordingly, in a first aspect, provided is a method for measuring heterodimerization of HIV RT, comprising:immobilizing a recombinant HIV RT subunit selected from a recombinant p51 subunit and a recombinant p66 subunit on a substrate, wherein each recombinant subunit comprises a tag and wherein the p51 subunit and the p66 subunit each comprise different tags; contacting the immodilized recombinant subunit with the other recombinant subunit thereby forming a heterodimer; contacting the heterodimer with an eTag covalently linked to an antibody that specifically binds to the heterodimer; and determining the amount of complex formed by cleaving the bound eTag and measuring the amount of cleaved eTag.

In certain embodiments, the recombinant p51 subunit has a wildtype amino acid sequence or mutant amino acid sequence. In certain embodiments, the recombinant p6 subunit has a wildtype amino acid sequence or a mutant amino acid sequence. In certain embodiments, the tag is selected from the group consisting of Histidine tag, c-myc tag, strep tag, calmodulin binding protein tag, substance P tag, the RYIRS tag, the Glu-Glu tag, CBD tag, E tag, GFP tag, GST tag, haemagglutinin tag, T7 tag, Tag 100, V5 tag, S tag, Intein/chitin binding domain tag, Xpress tag, thioredoxin tag, VSV tag and the FLAG tag. In certain embodiments, the tag is Histidine tag or FLAG tag. In certain embodiments, the recombinant p51 subunit comprises a Histidine tag and the recombinant p66 subunit comprises FLAG tag. In certain embodiments, the immobilizing comprises contacting Histidine-tagged p51 subunit homodimers to a Histidine-tag-binding substrate. In certain embodiments, cleaving the eTag comprises contacting the eTag with a photosensitizer or a chemi-activated sensitizer.

In another aspect, provided is a method for measuring heterodimerization of HIV RT, the method comprising:immobilizing His-tagged p51 subunit homodimers on a His-binding substrate; contacting the immobilized His-tagged p51 with a solution comprising FLAG-tagged p66 RT subunits and incubating under conditions that allow formation of p66/p51 RT subunits; contacting the p66/p51 RT subunits with eTags each covalently linked to an anti-FLAG antibody; and determining the amount of complex formed by cleaving the bound eTags and measuring the amount of cleaved eTags. In certain embodiments, cleaving the eTags comprises contacting the eTags with a photosensitizer or a chemi-activated sensitizer.

In another aspect, provided is a method for identifying compounds capable of modulating the HIV RT heterodimerization, the method comprising contacting immobilized His-tagged p51 subunits with a solution of FLAG-tagged p66 RT subunits in the presence or absence of a test compound capable of modulating the heterodimerization of HIV RT and incubating under conditions that allow formation of p66/p51 RT heterodimers; contacting the p66/p51 RT heterodimers with eTags each covalently linked to an anti-FLAG antibody; determining the amount of p66/p51 RT heterodimers formed by cleaving the bound eTags and measuring the amount of cleaved eTags; and comparing amount of p66/p51 RT heterodimers formed in the presence of the test compound sample to the amount of p66/p51 RT heterodimers formed in a control sample lacking the compound, whereby a decrease or an increase in p66/p51 RT heterodimer formation in the test compound sample is indicative of the ability of the compound to modulate heterodimerization. In certain embodiments, cleaving the eTags comprises contacting the eTags with a photosensitizer or a chemi-activated sensitizer.

In another aspect, provided is a method for measuring homodimerization of HIV RT, the method comprising contacting to homodimers of recombinant p51 subunit or recombinant p66 subunit, wherein the recombinant p51 subunit or recombinant p66 subunit comprises a tag, eTags each covalently linked to an anti-recombinant p51 subunit antibody or an anti-recombinant p66 subunit antibody; contacting to the homodimers a cleaving probe which binds the recombinant p51 subunit or the recombinant p66 subunit and has a cleavage-inducing moiety with an effective proximity, thereby bringing the eTag within the effective proximity of the cleaving probe releases the molecular tags, and determining the amount of complex formed by measuring the amount of cleaved eTags.

In certain embodiments, the recombinant p51 subunit has a wildtype amino acid sequence or mutant amino acid sequence. In certain embodiments, the recombinant p66 subunit has a wildtype amino acid sequence or mutant amino acid sequence. In certain embodiments, the tag is selected from the group consisting of Histidine tag, c-myc tag, strep tag, calmodulin binding protein tag, substance P tag, the RYIRS tag, the Glu-Glu tag, CBD tag, E tag, GFP tag, GST tag, haemagglutinin tag, T7 tag, Tag 100, V5 tag, S tag, Intein/chitin binding domain tag, Xpress tag, thioredoxin tag, VSV tag and the FLAG tag. In certain embodiments, the method further comprises inducing a sensitizer to generate an active species that cleaves the cleavable linkage(s) within the effective proximity. In certain embodiments, the sensitizer is a photosensitizer. The method further comprises illuminating the photosensitizer to generate an active species that cleaves the eTag(s) within the effective proximity. In certain embodiments, the active species is singlet oxygen. In certain embodiments, the cleaving probe comprises biotin. In certain embodiments, the method further comprises contacting the homodimers to streptavidin immobilized on a solid surface.

In another aspect, provided is a method for identifying compounds capable of modulating the HIV RT homodimerization, the method comprising forming homodimers of recombinant p51 subunit or recombinant p66 subunit, wherein the recombinant p51 subunit or recombinant p66 subunit comprises a tag, in the presence or absence of a test compound capable of modulating the homodimerization of HIV RT; contacting the homodimers wih eTags each covalently linked to an anti-recombinant p51 subunit antibody or an anti-recombinant p66 subunit antibody; contacting the homodimers with a cleaving probe which binds the recombinant p51 subunit or the recombinant p66 subunit and comprises a cleavage-inducing moiety with an effective proximity, thereby bringing the eTag within the effective proximity of the cleaving probe; and determining the amount of homodimers formed by cleaving the eTags and measuring the amount of cleaved eTags; and comparing the amount of homodimers in the presence of the test compound to the amount of homodimers formed in a control sample lacking the compound, whereby a decrease or an increase in homodimer formation in the test compound sample is indicative of the ability of the compound to modulate homodimerization. In certain embodiments, the cleaving probe comprises biotin. In certain embodiments, the method further comprises contacting the homodimers to streptavidin immobilized on a solid surface.

### BRIEF DESCRIPTION OF THE DRAWINGS

FIG. 1 illustrates the p66 and p51 expression vectors where p66 is FLAG-tagged while p51 is His-tagged.

FIG. 2 illustrates the heterodimer assay where a multi-welled His-binding plate can be contacted with His-tagged p51 polypeptide followed by the addition of FLAG-tagged p66 subunit. After enough time for the dimerization of p51 and p66 has elapsed, an eTag conjugated with an anti-FLAG antibody is added, the plate washed, and eTags released and quantitated.

FIG. 3 illustrates the effect of calmodulin (CAM), [2′,5′-bis-o-(butyldimethylsilyl)-3′-spiro 5′-(4′-amino-1′,2′-oxothiole-2′,2′-dioxide] (TSAO), and RAE family of proteins (RAE) on the dimerization of HIV RT.

FIG. 4 illustrates the effect of the efavirenz (EFV) on the dimerization of HIV RT.

FIG. 5 illustrates the homodimer assay where in each well either p51 or p66 can be incubated until homodimers form, and then scissor-conjugated biotinylated anti-RT antibody and eTag-conjugated anti-RT detector antibody can be added to the dimers. The dimers and antibody mix can be contacted with streptavidin coated plates, and scissors release eTags in close proximity. The released eTags can be detected and quantitated.

FIG. 6 illustrates the effect of EFV, Nevirapine (NVP), and CAM on the homodimerization of p66 and p 51 polypeptides of HIV RT.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF THE INVENTION

### Definitions

Unless otherwise stated, the following terms used in this application, including the specification and claims, have the definitions given below. It should be noted that, as used in the specification and the appended claims, the singular forms “a,” “an” and “the” include plural referents unless the context clearly dictates otherwise. Definition of standard chemistry terms may be found in reference works, including Carey and Sundberg (1992) “Advanced Organic Chemistry 3^{rd} Ed.” Vols. A and B, Plenum Press, New York. The methods will employ, unless otherwise indicated, conventional methods of synthetic organic chemistry, mass spectroscopy, preparative and analytical methods of chromatography, protein chemistry, biochemistry, recombinant DNA techniques and pharmacology, within the skill of the art.

The term “modulator” means a molecule that interacts with a target. The interactions include, but are not limited to, agonist, antagonist, and the like, as defined herein.

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proline: Pro (P) | Serine: Ser (S) |
| Threonine: Thr (T) | Tryptophan: Trp (W) |
| Tyrosine: Tyr (Y) | Valine: Val (V) |

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, as used herein, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations arising with hosts that produce the proteins or errors due to PCR amplification.

As used herein, an “analogue” or “derivative” is a compound, e.g., a peptide, having more than about 70% sequence but less than 100% sequence similarity with a given compound, e.g., a peptide. Such analogues or derivatives may be comprised of non-naturally occurring amino acid residues, including by way of example and not limitation, homoarginine, ornithine, penicillamine, and norvaline, as well as naturally occurring amino acid residues. Such analogues or derivatives may also be composed of one or a plurality of D-amino acid residues, and may contain non-peptide interlinkages between two or more amino acid residues.

As used herein, the terms “label”, “detectable label”, and “reporter molecule” refer to a molecule capable of being detected, including, but not limited to, radioactive isotopes, fluorescers, chemiluminescers, chromophores, magnetic resonance agents, enzymes, enzyme substrates, enzyme cofactors, enzyme inhibitors, chromophores, dyes, metal ions, metal sols, ligands (e.g., biotin, avidin, strepavidin or haptens) and the like. The term “fluorescer” refers to a substance or a portion thereof which is capable of exhibiting fluorescence in the detectable range.

“Antibody” means an immunoglobulin that specifically binds to, and is thereby defined as complementary with, a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgGG, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular polypeptide is maintained.

“Antibody binding composition” means a molecule or a complex of molecules that comprise one or more antibodies and derives its binding specificity from an antibody. Antibody binding compositions include, but are not limited to, antibody pairs in which a fist antibody binds specifically to a target molecule and a second antibody binds specifically to a constant region of the first antibody; a biotinylated antibody that binds specifically to a target molecule and streptavidin derivatized with moieties such as molecular tags or photosensitizers; antibodies specific for a target molecule and conjugated to a polymer, such as dextran, which, in turn, is derivatized with moieties such as molecular tags or photosensitizers; antibodies specific for a target molecule and conjugated to a bead, or microbead, or other solid phase support, which, in turn, is derivatized with moieties such as molecular tags or photosensitizers, or polymers containing the latter.

“Binding moiety” means any molecule to which molecular tags can be directly or indirectly attached that is capable of specifically binding to a membrane-associated analyte. Binding moieties include, but are not limited to, antibodies, antibody binding compositions, peptides, proteins, particularly secreted proteins and orphan secreted proteins, nucleic acids, and organic molecules having a molecular weight of up to 1000 daltons and consisting of atoms selected from the group consisting of hydrogen, carbon, oxygen, nitrogen, sulfur, and phosphorus.

“Chromatography” or “chromatographic separation” as used herein means or refers to a method of analysis in which the flow of a mobile phase, usually a liquid, containing a mixture of compounds, e.g. molecular tags, promotes the separation of such compounds based on one or more physical or chemical properties by a differential distribution between the mobile phase and a stationary phase, usually a solid. The one or more physical characteristics that form the basis for chromatographic separation of analytes, such as molecular tags, include but are not limited to molecular weight, shape, solubility, pKa, hydrophobicity, charge, polarity, and the like. In one aspect, as used herein, “high pressure (or performance) liquid chromatography” (“HPLC”) refers to a liquid phase chromatographic separation that (i) employs a rigid cylindrical separation column having a length of up to 300 mm and an inside diameter of up to 5 mm, (ii) has a solid phase comprising rigid spherical particles (e.g. silica, alumina, or the like) having the same diameter of up to 5 Wn packed into the separation column, (iii) takes place at a temperature in the range of from 35° C. to 80° C. and at column pressure up to 150 bars, and (iv) employs a flow rate in the range of from 1 μL/min to 4 mL/min. Preferably, solid phase particles for use in HPLC are further characterized in (i) having a narrow size distribution about the mean particle diameter, with substantially all particle diameters being within 10% of the mean, (ii) having the same pore size in the range of from 70 to 300 angstroms, (iii) having a surface area in the range of from 50 to 250 m²/g, and (iv) having a bonding phase density (i.e. the number of retention ligands per unit area) in the range of from 1 to 5 per nm². Exemplary reversed phase chromatography media for separating molecular tags include particles, e.g. silica or alumina, having bonded to their surfaces retention ligands, such as phenyl groups, cyano groups, or aliphatic groups selected from the group including C₈ through C₁₈. Chromatography includes “capillary electrochromatography” (“CEC”), and related techniques. CEC is a liquid phase chromatographic technique in which fluid is driven by electroosmotic flow through a capillary-sized column, e.g. with inside diameters in the range of from 30 to 100 μm. CEC is disclosed in Svec, Adv. Biochem. Eng. Biotechnol. 76: 1 47 (2002); Vanhoenacker et al, Electrophoresis, 22: 4064 4103 (2001); and like references. CEC column may use the same solid phase materials as used in conventional reverse phase HPLC and additionally may use so-called “monolithic” non-particular packings. In some forms of CEC, pressure as well as electroosmosis drives an analyte-containing solvent through a column.

The term “sample” means a quantity of material that is suspected of containing or known to contain analytes that are to be detected or measured. As used herein, the term includes a specimen (e.g., a biopsy or medical specimen) or a culture (e.g., microbiological culture). It also includes both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types. In particular, biological samples include fixed biological specimens, such as patient biopsy specimens treated with a fixative, biological specimens embedded in paraffin, frozen biological specimens, smears, and the like.

A “separation profile” in reference to the separation of molecular tags means a chart, graph, curve, bar graph, or other representation of signal intensity data versus a parameter related to the molecular tags, such as retention time, mass, or the like, that provides a readout, or measure, of the number of molecular tags of each type produced in an assay. A separation profile may be an electropherogram, a chromatogram, an electrochromatogram, a mass spectrogram, or like graphical representation of data depending on the separation technique employed. A “peak” or a “band” or a “zone” in reference to a separation profile means a region where a separated compound is concentrated. There may be multiple separation profiles for a single assay if, for example, different molecular tags have different fluorescent labels having distinct emission spectra and data is collected and recorded at multiple wavelengths. In one aspect, released molecular tags are separated by differences in electrophoretic mobility to form an electropherogram wherein different molecular tags correspond to distinct peaks on the electropherogram. A measure of the distinctness, or lack of overlap, of adjacent peaks in an electropherogram is “electrophoretic resolution,” which may be taken as the distance between adjacent peak maximums divided by four times the larger of the two standard deviations of the peaks. Preferably, adjacent peaks have a resolution of at least 1.0, and more preferably, at least 1.5, and most preferably, at least 2.0. In a given separation and detection system, the desired resolution may be obtained by selecting a plurality of molecular tags whose members have electrophoretic mobilities that differ by at least a peak-resolving amount, such quantity depending on several factors well known to those of ordinary skill, including signal detection system, nature of the fluorescent moieties, the diffusion coefficients of the tags, the presence or absence of sieving matrices, nature of the electrophoretic apparatus, e.g. presence or absence of channels, length of separation channels, and the like.

“Specific” or “specificity” in reference to the binding of one molecule to another molecule, such as a binding compound, or probe, for a target analyte, means the recognition, contact, and formation of a stable complex between the probe and target, together with substantially less recognition, contact, or complex formation of the probe with other molecules. In one aspect, “specific” in reference to the binding of a first molecule to a second molecule means that to the extent the first molecule recognizes and forms a complex with another molecules in a reaction or sample, it forms the largest number of the complexes with the second molecule. In one aspect, this largest number is at least fifty percent of all such complexes form by the first molecule. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, ionic and hydrophobic interactions, and the like, dominate the interaction of the molecules. As used herein, “stable complex” in reference to two or more molecules means that such molecules form noncovalently linked aggregates, e.g. by specific binding, that under assay conditions are thermodynamically more favorable than a non-aggregated state.

As used herein, the term “spectrally resolvable” in reference to a plurality of fluorescent labels means that the fluorescent emission bands of the labels are sufficiently distinct, ie. sufficiently non-overlapping, that molecular tags to which the respective labels are attached can be distinguished on the basis of the fluorescent signal generated by the respective labels by standard photodetection systems, e.g. employing a system of band pass filters and photomultiplier tubes, or the like, as exemplified by the systems described in U.S. Pat. Nos. 4,230,558; 4,811,218, or the like, or in Wheeless et al, pgs. 21 76, in Flow Cytometry: Instrumentation and Data Analysis (Academic Press, New York, 1985).

The term “tag” is used herein to denote a peptide or polypeptide segment, or other moiety, that can be attached to a second polypeptide to provide detection of the second polypeptide or provide sites for attachment of the second polypeptide to a substrate. In principal, any peptide or protein for which an antibody or other specific binding agent is available can be used as an tag. Tags include a poly-histidine tract, protein A, glutathione S transferase, Glu-Glu affinity tag, substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). DNAs encoding affinity tags are available from commercial suppliers (e.g., Pharmacia Biotech, Piscataway, N.J.).

As used herein, the term “tagged probe” refers to a probe that binds to a target molecule on the surface of a cell membrane, i.e. membrane-associated analyte, and which comprises one or more molecular tags linked to a binding agent of the probe through a cleavable linkage. As used herein, “tagged probe” is used synonymously with ‘binding compound.”

As used herein, the term “wild-type” refers to an HIV-1 gene or HIV-1 protein encoded by the HIV-1 gene that has the nucleic acid sequence of reference HIV-1 strain NL4-3 (GenBank Accession No. AF324493).

### Methods for Determining the Presence of Amount of HIV RT Dimers or Oligomers

Provided herein are methods for determining the presence and/or amount of dimers or oligomers of one or more subunits of HIV reverse transcriptase (HIV RT) in a sample by selectively releasing molecular tags from binding compounds that form stable complexes together with the homo- and heterodimers of p66 and p51 subunits of HIV RT and a cleaving probe.

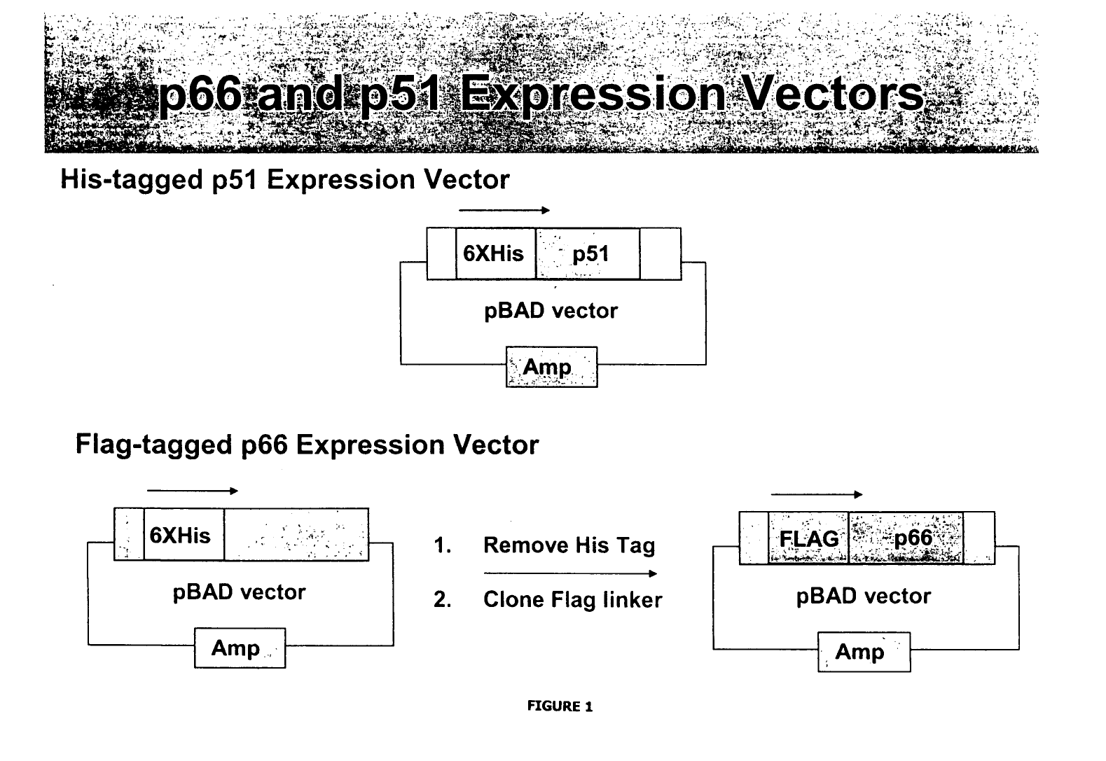

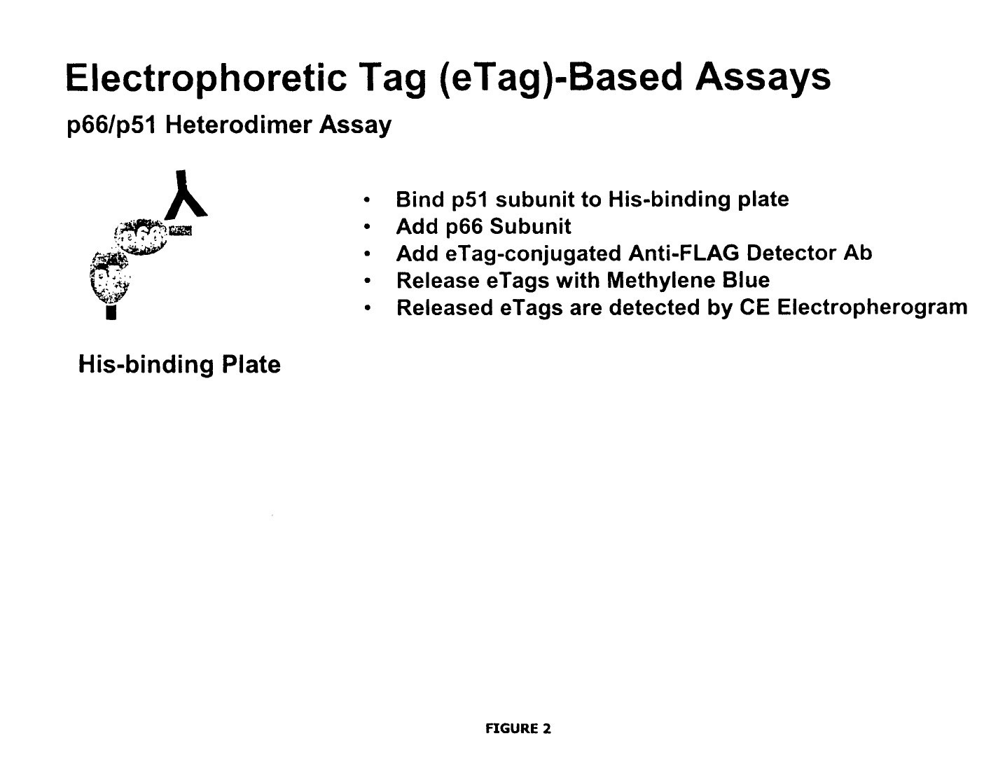

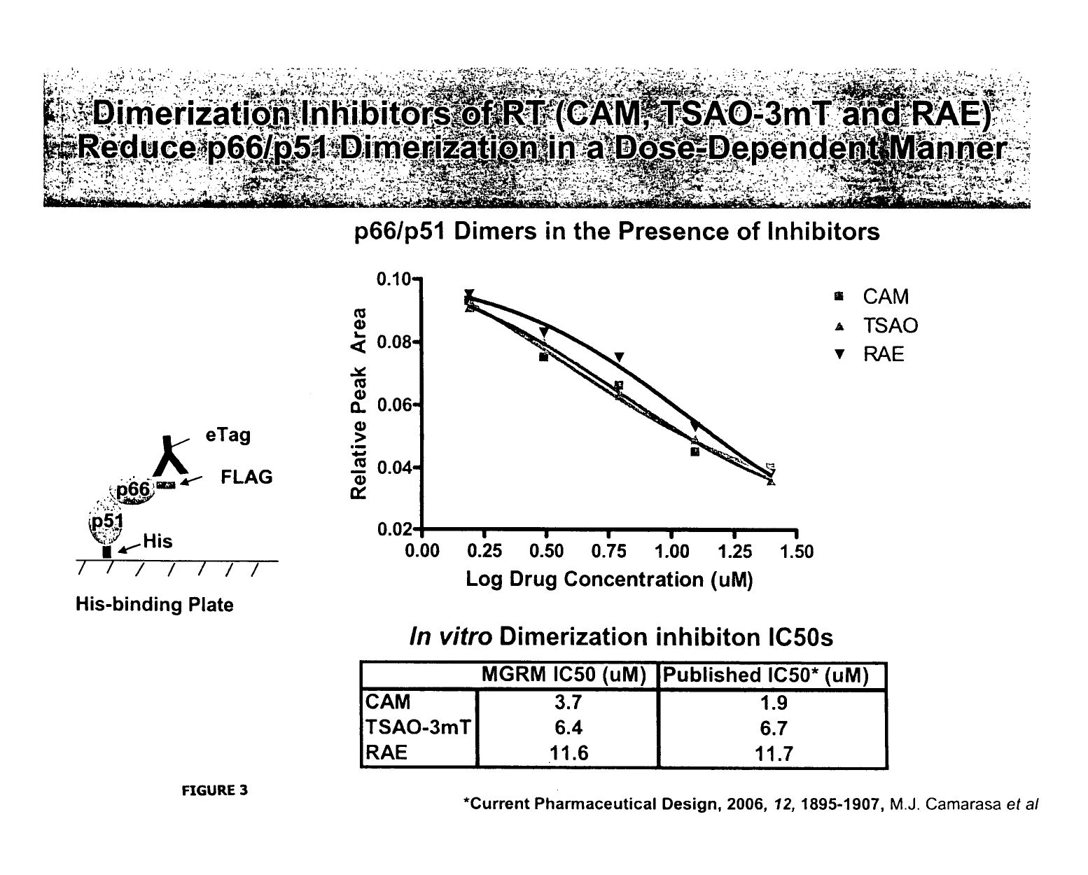

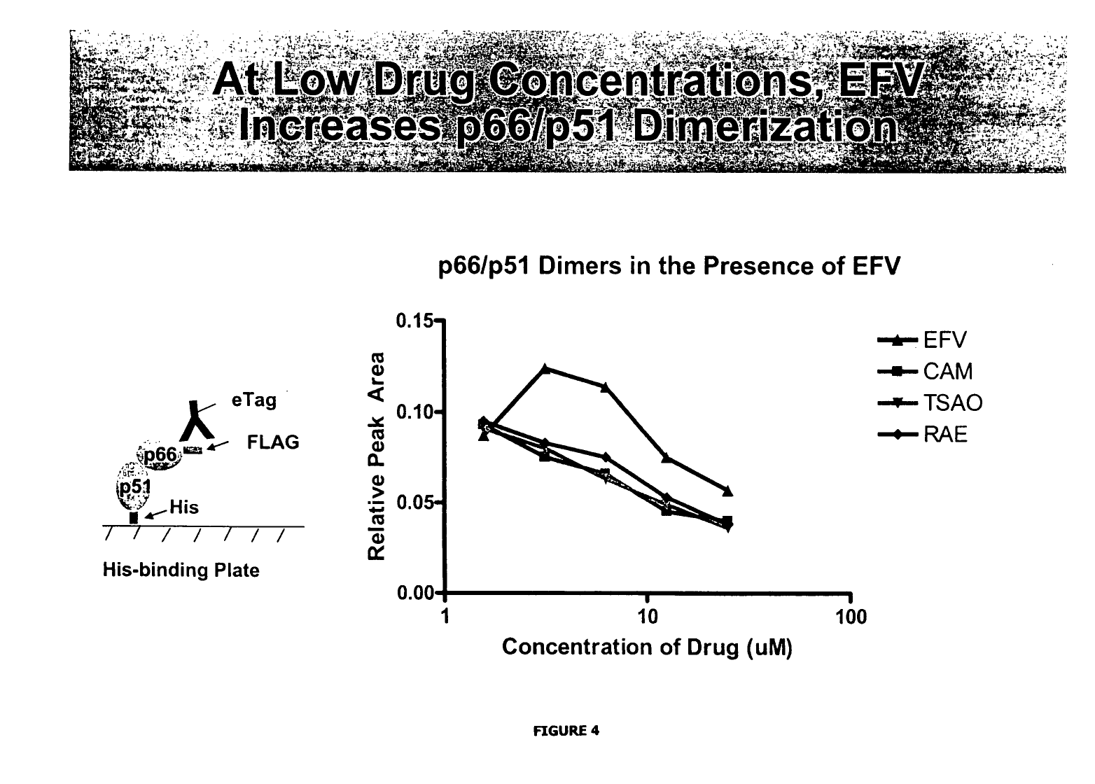

In one aspect, assays for determining the heterodimerization of p51 and p66 subunits of HIV RT are provided. The p51 subunit can be labeled with a tag, such as polyHis tag, and the p66 subunit can be labeled with a different tag, such as FLAG. The tagging can be done using pBAD/FLAG or pBAD/His expression vectors that are commercially available, and bacterially expressing and purifying the tagged subunits (FIG. 1). The His-tagged subunit can be contacted with a His-binding plate thereby immobilizing the His-tagged subunit to the plate (FIG. 2). Then the differently labeled second subunit can be added. To the heterodimer complex that forms can be added eTag-conjugated anti-FLAG antibody. The plate can be washed, and the eTags released and the released eTags can be quantitated. The binding of the subunits can be performed in the presence or absence of drugs to determine the effect of the drugs on the heterodimerization of the p51 and p66 subunits of HIV RT (FIGS. 3 and 4).

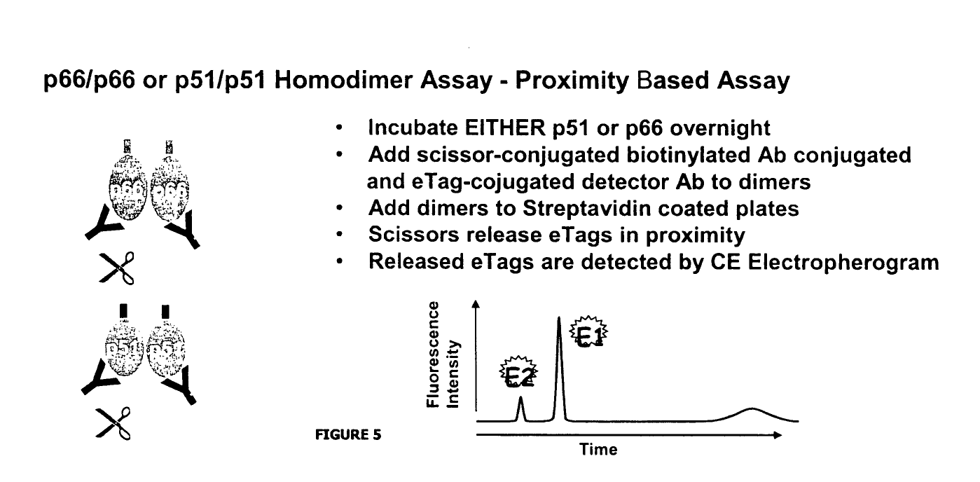

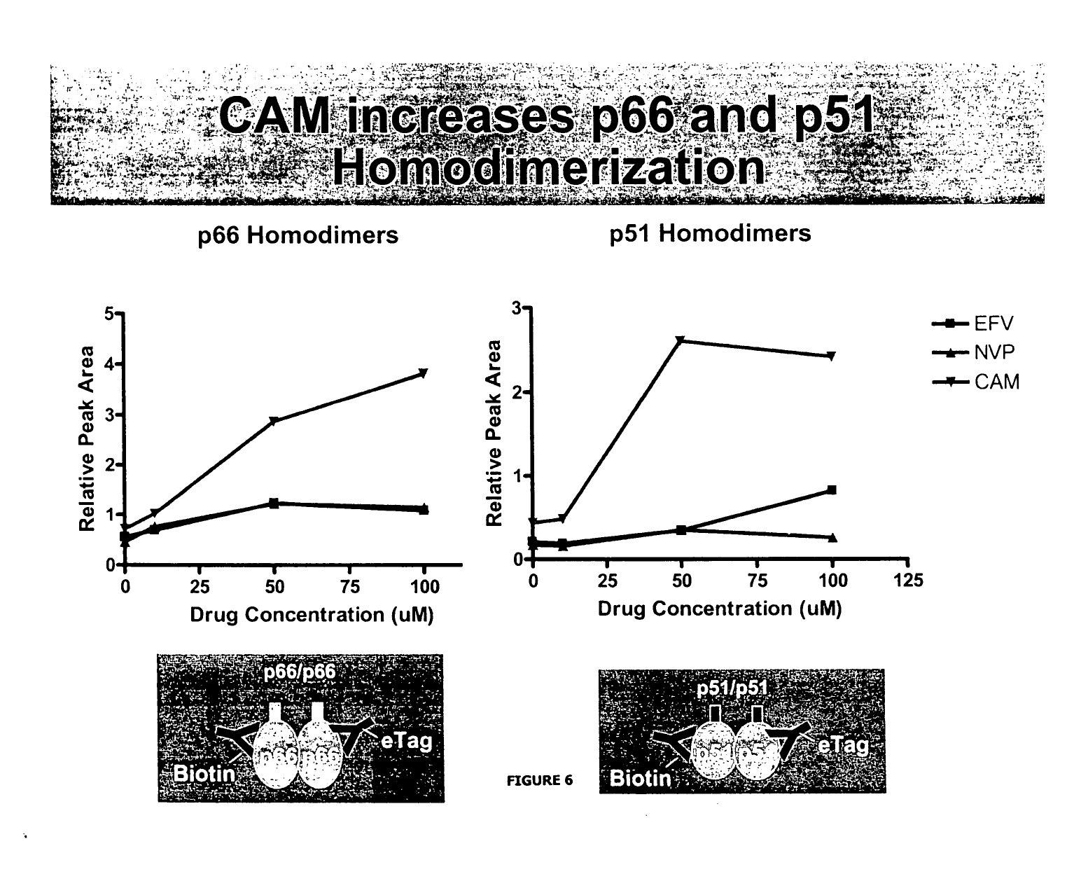

In another aspect, an assay for determining the homodimerization of p51 and p66 subunits of HIV RT is provided (FIG. 5). The p51 subunit or the p66 subunit can be incubated until homodimers form. To the homodimer complex can be added scissor-conjugated biotinylated anti-RT antibody that binds to either the p51 or the p66 subunits and eTag-conjugated anti-RT detector antibody where the second antibody binds to the same subunit. The dimers and antibody mix can be contacted with Streptavidin coated plates, and scissors release eTags in close proximity. The released eTags can be detected and quantitated. The homodimerization of the subunits can be performed in the presence or absence of drugs to determine the effect of the drugs on the homodimerization of the p51 or the p66 subunits of HIV RT (FIG. 6). One of skill in the art will recognize that this assay can also be performed to determine heterodimerization of HIV RT. In such embodiments, the antibody of the scissor containing reagent can be selected to bind to one of the subunits (for example, p51) and the antibody of the eTag-containing reagent can be select to bind to the other subunit, for example, p66.

### Compositions Comprising p66 and p51 Polyeptides

One aspect includes p66 and p51 recombinant proteins or nucleic acids encoding these molecules. For instance, the recombinant or fusion protein comprises the p66 or the p51 subunits and amino acids to form a “tag.” Such tags include, but are not limited to, His, FLAG, Myc and GST. The tags can be added to the C-terminus, N-terminus, or within the amino acid sequence of the p66 and p51 proteins.

Peptide tags include, for example, Histidine tags, c-myc tags, strep tag, calmodulin binding protein, substance P, the RYIRS tag, the Glu-Glu tag, CBD tag, E tag, GFP tag, GST tag, haemagglutinin tag, T7 tag, Tag 100, V5 tag, S tag, Intein/chitin binding domain tag, Xpress tag, thioredoxin tag, VSV tag and the FLAG tag. See, for example, Morganti et al., 1996 Biotechnol. Appl. Biochem. 23:67. Nucleic acid molecules encoding such peptide tags are available, for example, from Sigma-Aldrich Corporation (St. Louis, Mo.).

In one aspect, the recombinant peptides comprise a fusion of the subunits of HIV RT with a tag polypeptide which provides an epitope to which an anti-tag antibody can selectively bind. The epitope tag can generally be placed at the amino- or carboxyl-terminus of the RT subunits. The presence of such epitope-tagged forms of the recombinant peptide can be detected using an antibody against the tag polypeptide.

Alternately, any non-peptide tags known to one skilled in the art could also be used.

### Molecular Tags and Cleavable Linkages

In an aspect, molecular tags are cleaved from a binding compound, or tagged probe, by reaction of a cleavable linkage with an active species, such as singlet oxygen, generated by a cleavage-inducing moiety. For example, Singh et al, International patent publications WO 01/83502 and WO 02/95356 disclose numerous suitable molecular tags, cleavable linkers, and cleavage-inducing moieties.

In one aspect, provided are mixtures of pluralities of different binding compounds, wherein each different binding compound has one or more molecular tags attached through cleavable linkages. The nature of the binding compound, cleavable linkage and molecular tag may vary widely. A binding compound may comprise an antibody binding composition, an antibody, a peptide, a peptide or non-peptide ligand for a cell surface receptor, a protein, an oligonucleotide, an oligonucleotide analog, such as a peptide nucleic acid, a lectin, or any other molecular entity that is capable of specific binding or complex formation with a membrane-associated analyte of interest. Preferably, antibodies to FLAG, the p51 subunit or the p66 subunit are used. In one aspect, a binding compound, which can be represented by the formula below, comprises one or more molecular tags attached to an analyte-specific binding moiety.

B-(L-E)ₖ

wherein B is a binding moiety; L is a cleavable linkage; and E is a molecular tag. Preferably, in homogeneous assays for non-polynucleotide analytes, cleavable linkage, L, is an oxidation-labile linkage, and more preferably, it is a linkage that may be cleaved by singlet oxygen. The moiety “(L-E)ₖ” indicates that a single binding compound may have multiple molecular tags attached via cleavable linkages. In one aspect, k is an integer greater than or equal to one, but in other embodiments, k may be greater than several hundred, e.g. 100 to 500, or k is greater than several hundred to as many as several thousand, e.g. 500 to 5000. Within a composition, usually each of the plurality of different types of binding compound has a different molecular tag, E. Cleavable linkages, e.g. oxidation-labile linkages, and molecular tags, E, are attached to B by way of conventional chemistries.

Preferably, B is an antibody binding composition. Such compositions are readily formed from a wide variety of commercially available antibodies, both monoclonal and polyclonal, specific for membrane-associated analytes. preferably, the antibodies are for FLAG, the p51 subunit, or the p66 subunit.

When L is oxidation labile, L is preferably a thioether or its selenium analog; or an olefin, which contains carbon-carbon double bonds, wherein cleavage of a double bond to an oxo group, releases the molecular tag, E. Illustrative thioether bonds are disclosed in Willner et al, U.S. Pat. No. 5,622,929. Illustrative olefins include vinyl sulfides, vinyl ethers, enamines, imines substituted at the carbon atoms with an α-methine (CH, a carbon atom having at least one hydrogen atom), where the vinyl group may be in a ring, the heteroatom may be in a ring, or substituted on the cyclic olefinic carbon atom, and there will be at least one and up to four heteroatoms bonded to the olefinic carbon atoms. The resulting dioxetane may decompose spontaneously, by heating above ambient temperature, usually below about 75° C., by reaction with acid or base, or by photo-activation in the absence or presence of a photosensitizer. Such reactions are described in the following exemplary references: Adam and Liu, J. Amer. Chem. Soc. 94, 1206 1209,1972, Ando, et al., J. C. S. Chem. Comm. 1972,477 8, Ando, et al., Tetrahedron 29, 1507 13, 1973, Ando, et al., J. Amer. Chem. Soc. 96, 6766 8, 1974, Ando and Migita, ibid. 97, 5028 9,1975, Wasserman and Terao, Tetra. Lett. 21, 1735 38, 1975, Ando and Watanabe, ibid. 47, 4127 30, 1975, Zaklika, et al., Photochemistry and Photobiology 30, 3544, 1979, and Adam, et al., Tetra. Lett. 36, 7853 4, 1995. See also, U.S. Pat. No. 5,756,726.

The formation of dioxetanes is obtained by the reaction of singlet oxygen with an activated olefin substituted with an molecular tag at one carbon atom and the binding moiety at the other carbon atom of the olefin. See, for example, U.S. Pat. No. 5,807,675. These cleavable linkages may be depicted by the following formula:

—W—(X)ₙC_{α}═C_{β}(y)(Z)—

wherein:

W may be a bond, a heteroatom, e.g., O, S, N, P, M (intending a metal that forms a stable covalent bond), or a functionality, such as carbonyl, imino, etc., and may be bonded to X or C a at least one X will be aliphatic, aromatic, alicyclic or heterocyclic and bonded to C, (through a hetero atom, e.g., N, O, or S and the other X may be the same or different and may in addition be hydrogen, aliphatic, aromatic, alicyclic or heterocyclic, usually being aromatic or aromatic heterocyclic wherein one X may be taken together with Y to form a ring, usually a heterocyclic ring, with the carbon atoms to which they are attached, generally when other than hydrogen being from about 1 to 20, usually 1 to 12, more usually 1 to 8 carbon atoms and one X will have 0 to 6, usually 0 to 4 heteroatoms, while the other X will have at least one heteroatom and up to 6 heteroatoms, usually 1 to 4 heteroatoms;

Y will come within the definition of X, usually being bonded to C through a heteroatom and as indicated may be taken together with X to form a heterocyclic ring;

Z will usually be aromatic, including heterocyclic aromatic, of from about 4 to 12, usually 4 to 10 carbon atoms and 0 to 4 heteroatoms, as described above, being bonded directly to C_{β} or through a heteroatom, as described above;

n is 1 or 2, depending upon whether the molecular tag is bonded to C_{α} or X;

wherein one of Y and Z will have a functionality for binding to the binding moiety, or be bound to the binding moiety, e.g. by serving as, or including a linkage group, to a binding moiety, T.

Preferably, W, X, Y, and Z are selected so that upon cleavage molecular tag, E, is within the size limits described below.

Illustrative cleavable linkages include S(molecular tag)-3-thiolacrylic acid, N(molecular tag), N-methyl 4-amino-4-butenoic acid, 3-hydroxyacrolein, N-(4-carboxyphenyl)-2-(molecular tag)-imidazole, oxazole, and thiazole.

Also of interest are N-alkyl acridinyl derivatives, substituted at the 9 position with a divalent group of the formula:

—(CO)X¹(A)-

wherein:

X¹ is a heteroatom selected from the group consisting of O, S, N, and Se, usually one of the first three; and

A is a chain of at least 2 carbon atoms and usually not more than 6 carbon atoms substituted with an molecular tag, where preferably the other valences of A are satisfied by hydrogen, although the chain may be substituted with other groups, such as alkyl, aryl, heterocyclic groups, etc., A generally being not more than 10 carbon atoms.

Also of interest are heterocyclic compounds, such as diheterocyclopentadienes, as exemplified by substituted imidazoles, thiazoles, oxazoles, etc., where the rings will usually be substituted with at least one aromatic group and in some instances hydrolysis will be used to release the molecular tag.

Also of interest are tellurium (Te) derivatives, where the Te is bonded to an ethylene group having a hydrogen atom, wherein the ethylene group is part of an alicyclic or heterocyclic ring, that may have an oxo group, preferably fused to an aromatic ring and the other valence of the Te is bonded to the molecular tag. The rings may be coumarin, benzoxazine, tetralin, etc.

Several preferred cleavable linkages and their cleavage products are procided in U.S. Pat. No. 7,105,308, and include thioether cleavable linkages shown below:

formula:

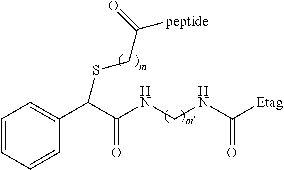

wherein:
m and m′ are integers independently chosen from 1 to 10.
Molecular tag, E, may comprise an electrophoric tag as described in the following references when separation of pluralities of molecular tags are carried out by gas chromatography or mass spectrometry: Zhang et al, Bioconjugate Chem., 13: 1002 1012 (2002); Giese, Anal. Chem., 2: 165 168 (1983); and U.S. Pat. Nos. 4,650,750; 5,360,819; 5,516,931; 5,602,273; and the like.

Molecular tag, E, is preferably a water-soluble organic compound that is stable with respect to the active species, especially singlet oxygen, and that includes a detection or reporter group. Otherwise, E may vary widely in size and structure. In one aspect, E has a molecular weight in the range of from about 50 to about 2500 daltons, more preferably, from about 50 to about 1500 daltons. Preferred structures of E are described more fully below. E may comprise a detection group for generating an electrochemical, fluorescent, or chromogenic signal. In embodiments employing detection by mass, E may not have a separate moiety for detection purposes. Preferably, the detection group generates a fluorescent signal.

Molecular tags within a plurality are selected so that each has a unique separation characteristic and/or a unique optical property with respect to the other members of the same plurality. In one aspect, the chromatographic or electrophoretic separation characteristic is retention time under set of standard separation conditions conventional in the art, e.g. voltage, column pressure, column type, mobile phase, electrophoretic separation medium, or the like. In another aspect, the optical property is a fluorescence property, such as emission spectrum, fluorescence lifetime, fluorescence intensity at a given wavelength or band of wavelengths, or the like. Preferably, the fluorescence property is fluorescence intensity. For example, each molecular tag of a plurality may have the same fluorescent emission properties, but each will differ from one another by virtue of a unique retention time. On the other hand, or two or more of the molecular tags of a plurality may have identical retention times, but they will have unique fluorescent properties, e.g. spectrally resolvable emission spectra, so that all the members of the plurality are distinguishable by the combination of molecular separation and fluorescence measurement.

Preferably, released molecular tags are detected by electrophoretic separation and the fluorescence of a detection group. In such embodiments, molecular tags having substantially identical fluorescence properties have different electrophoretic mobilities so that distinct peaks in an electropherogram are formed under separation conditions. Preferably, pluralities of molecular tags are separated by conventional capillary electrophoresis apparatus, either in the presence or absence of a conventional sieving matrix. Exemplary capillary electrophoresis apparatus include Applied Biosystems (Foster City, Calif.) models 310, 3100 and 3700; Beckman (Fullerton, Calif.) model P/ACE MDQ; Amersham Biosciences (Sunnyvale, Calif.) MegaBACE 1000 or 4000; SpectruMedix genetic analysis system; and the like. Electrophoretic mobility is proportional to q/M²/3, where q is the charge on the molecule and M is the mass of the molecule. Desirably, the difference in mobility under the conditions of the determination between the closest electrophoretic labels will be at least about 0.001, usually 0.002, more usually at least about 0.01, and may be 0.02 or more. Preferably, in such conventional apparatus, the electrophoretic mobilities of molecular tags of a plurality differ by at least one percent, and more preferably, by at least a percentage in the range of from 1 to 10 percent.

In one aspect, molecular tag, E, is (M, D), where M is a mobility-modifying moiety and D is a detection moiety. The notation “(M, D)” is used to indicate that the ordering of the M and D moieties may be such that either moiety can be adjacent to the cleavable linkage, L. That is, “B-L-(M, D)” designates binding compound of either of two forms: “B-L-M-D” or “B-L-D-M.” Detection moiety, D, may be a fluorescent label or dye, a chromogenic label or dye, an electrochemical label, or the like. Preferably, D is a fluorescent dye. Exemplary fluorescent dyes include water-soluble rhodamine dyes, fluoresceins, 4,7-dichlorofluoresceins, benzoxanthene dyes, and energy transfer dyes. Further specific exemplary fluorescent dyes include 5- and 6-carboxyrhodamine 6G; 5- and 6-carboxy-X-rhodamine, 5- and 6-carboxytetramethylrhodamine, 5- and 6-carboxyfluorescein, 5- and 6-carboxy-4,7-dichlorofluor 2′,7′-irethoxy-5- and 6-carboxy-4,7-dichlorofluorescein, 2′,7′-dimethoxy-4′,5′-dichloro-6-carboxy-4,7-dichlorofluorescein, 1′,2′,7′,8′-dibenzo-5- and 6-carboxy-4,7-dichlorofluorescein, 1′,2′,7′,8′-dibenzo4′,5′-dichloro-5- and 6-carboxy-4,7-dichlorofluorescein, 2′,7′-d 6-carboxy-4,7-dichlorofluorescein, and 2′,4′,5′,7′-tetrachloro-5- and 6-carboxy-4,7-dichlorofluor. Most preferably, D is a fluorescein or a fluorescein derivative.

The size and composition of mobility-modifying moiety, M, can vary from a bond to about 100 atoms in a chain, usually not more than about 60 atoms, more usually not more than about 30 atoms, where the atoms are carbon, oxygen, nitrogen, phosphorous, boron and sulfur. Generally, when other than a bond, the mobility-modifing moiety has from about 0 to about 40, more usually from about 0 to about 30 heteroatoms, which in addition to the heteroatoms indicated above may include halogen or other heteroatom. The total number of atoms other than hydrogen is generally fewer than about 200 atoms, usually fewer than about 100 atoms. Where acid groups are present, depending upon the pH of the medium in which the mobility-modifying moiety is present, various cations may be associated with the acid group. The acids may be organic or inorganic, including carboxyl, thionocarboxyl, thiocarboxyl, hydroxamic, phosphate, phosphite, phosphonate, phosphinate, sulfonate, sulfinate, boronic, nitric, nitrous, etc. For positive charges, substituents include amino (includes ammonium), phosphonium, sulfonium, oxonium, etc., where substituents are generally aliphatic of from about 16 carbon atoms, the total number of carbon atoms per heteroatom, usually be less than about 12, usually less than about 9. The side chains include amines, ammonium salts, hydroxyl groups, including phenolic groups, carboxyl groups, esters, amides, phosphates, heterocycles. M may be a homo-oligomer or a hetero-oligomer, having different monomers of the same or different chemical characteristics, e.g., nucleotides and amino acids.

M may also comprise polymer chains prepared by known polymer subunit synthesis methods. Methods of forming selected-length polyethylene oxide-containing chains are well known, e.g. Grossman et al, U.S. Pat. No. 5,777,096. It can be appreciated that these methods, which involve coupling of defined-size, multi-subunit polymer units to one another, directly or via linking groups, are applicable to a wide variety of polymers, such as polyethers (e.g., polyethylene oxide and polypropylene oxide), polyesters (e.g., polyglycolic acid, polylactic acid), polypeptides, oligosaccharides, polyurethanes, polyamides, polysulfonamides, polysulfoxides, polyphosphonates, and block copolymers thereof, including polymers composed of units of multiple subunits linked by charged or uncharged linking groups. In addition to homopolymers, the polymer chains include selected-length copolymers, e.g., copolymers of polyethylene oxide units alternating with polypropylene units. As another example, polypeptides of selected lengths and amino acid composition (ie., containing naturally occurring or man-made amino acid residues), as homopolymers or mixed polymers.

In another aspect, after release, molecular tag, E, is defined by the formula:

A-M-D

wherein:

A is —C(═O)R, where R is aliphatic, aromatic, alicyclic or heterocyclic having from 1 to 8 carbon atoms and 0 to 4 heteroatoms selected from the group consisting of O, S, and N; —CH₂—C(═O)—NH—CHO; —SO₂H; —CH₂—C(═O)O—CHO; —C(═O)NH—(CH₂)ₙ—NH—C(═O)C(═O)— where n is in the range of from 2 to 12;

D is a detection group, preferably a fluorescent dye; and

M is as described above, with the proviso that the total molecular weight of A-M-D be within the range of from about 100 to about 2500 daltons.

In another aspect, D is a fluorescein and the total molecular weight of A-M-D is in the range of from about 100 to about 1500 daltons.

In another aspect, M may be synthesized from smaller molecules that have functional groups that provide for linking of the molecules to one another, usually in a linear chain. Such functional groups include carboxylic acids, amines, and hydroxy- or thiol-groups. The charge-imparting moiety may have one or more side groups pending from the core chain. The side groups have a functionality to provide for linking to a label or to another molecule of the charge-imparting moiety. Common functionalities resulting from the reaction of the functional groups employed are exemplified by forming a covalent bond between the molecules to be conjugated. Such functionalities are disulfide, amide, thioamide, dithiol, ether, urea, thiourea, guanidine, azo, thioether, carboxylate and esters and amides containing sulfur and phosphorus such as, e.g., sulfonate, phosphate esters, sulfonamides, thioesters, etc., and the like.

### Attaching Molecular Tags to Binding Moieties

Extensive guidance can be found in the literature for covalently linking molecular tags to binding compounds, such as antibodies, e.g. Hermanson, Bioconjugate Techniques, (Academic Press, New York, 1996), and the like. In one aspect, one or more molecular tags are attached directly or indirectly to common reactive groups on a binding compound. Common reactive groups include amine, thiol, carboxylate, hydroxyl, aldehyde, ketone, and the like, and may be coupled to molecular tags by commercially available cross-linking agents, e.g. Hermanson (cited above); Haugland, Handbook of Fluorescent Probes and Research Products, Ninth Edition (Molecular Probes, Eugene, Oreg., 2002). In one embodiment, an NHS-ester of a molecular tag is reacted with a free amine on the binding compound.

In one aspect, binding compounds comprise a biotinylated antibody (140) as a binding moiety. Molecular tags are attached to binding moiety by way of avidin or streptavidin bridge. Preferably, in operation, binding moiety is first reacted with membrane-bound analytes, after which avidin or streptavidin is added to form complex. To complexes can be added biotinylated molecular tags to form binding compound.

Once each of the binding compounds is separately derivatized by a different molecular tag, it is pooled with other binding compounds to form a plurality of binding compounds. Usually, each different kind of binding compound is present in a composition in the same proportion; however, proportions may be varied as a design choice so that one or a subset of particular binding compounds are present in greater or lower proportion depending on the desirability or requirements for a particular embodiment or assay. Factors that may affect such design choices include, but are not limited to, antibody affinity and avidity for a particular target, relative prevalence of a target, fluorescent characteristics of a detection moiety of a molecular tag, and the like.

### Cleavage-Inducing Moiety Producing Active Species

A cleavage-inducing moiety, or cleaving agent, is a group that produces an active species that is capable of cleaving a cleavable linkage, preferably by oxidation. Preferably, the active species is a chemical species that exhibits short-lived activity so that its cleavage-inducing effects are only in the proximity of the site of its generation. Either the active species is inherently short lived, so that it will not create significant background because beyond the proximity of its creation, or a scavenger is employed that efficiently scavenges the active species, so that it is not available to react with cleavable linkages beyond a short distance from the site of its generation. Illustrative active species include singlet oxygen, hydrogen peroxide, NADH, and hydroxyl radicals, phenoxy radical, superoxide, and the like. Illustrative quenchers for active species that cause oxidation include polyenes, carotenoids, vitamin E, vitamin C, amino acid-pyrrole N-conjugates of tyrosine, histidine, and glutathione, and the like, e.g. Beutner et al, Meth. Enzymol., 319: 226 241 (2000).

In one aspect, the cleavage-inducing moiety and the cleavable linkage are in close proximity or not be so far removed from one another when bound to a target protein that the active species generated by the sensitizer diffises and loses its activity before it can interact with the cleavable linkage. Accordingly, a cleavable linkage preferably are within 1000 nm, preferably 20-200 nm of a bound cleavage-inducing moiety. This effective range of a cleavage-inducing moiety is referred to herein as its “effective proximity.”

Generators of active species include enzymes, such as oxidases, such as glucose oxidase, xanthene oxidase, D-amino acid oxidase, NADH-FMN oxidoreductase, galactose oxidase, glyceryl phosphate oxidase, sarcosine oxidase, choline oxidase and alcohol oxidase, that produce hydrogen peroxide, horse radish peroxidase, that produces hydroxyl radical, various dehydrogenases that produce NADH or NADPH, urease that produces ammonia to create a high local pH.

A sensitizer is a compound that can be induced to generate a reactive intermediate, or species, usually singlet oxygen. Preferably, the sensitizer is a photosensitizer. Other sensitizers can be compounds that on excitation by heat, light, ionizing radiation, or chemical activation will release a molecule of singlet oxygen. The best known members of this class of compounds include the endoperoxides such as 1,4-biscarboxyethyl-1,4-naphthalene endoperoxide, 9,10-diphenylanthracene-9,10-endoperoxide and 5,6,11,12-tetraphenyl naphthalene 5,12-endoperoxide. Heating or direct absorption of light by these compounds releases singlet oxygen.

Attachment of a binding agent to the cleavage-inducing moiety may be direct or indirect, covalent or non-covalent and can be accomplished by well-known techniques, commonly available in the literature. See, for example, “Immobilized Enzymes,” Ichiro Chibata, Halsted Press, New York (1978); Cuatrecasas, J. Biol. Chem., 245:3059 (1970). A wide variety of functional groups are available or can be incorporated. Functional groups include carboxylic acids, aldehydes, amino groups, cyano groups, ethylene groups, hydroxyl groups, mercapto groups, and the like. The manner of linking a wide variety of compounds is well known and is amply illustrated in the literature (see above). The length of a linking group to a binding agent may vary widely, depending upon the nature of the compound being linked, the effect of the distance on the specific binding properties and the like.

It may be desirable to have multiple cleavage-inducing moieties attached to a binding agent to increase, for example, the number of active species generated. This can be accomplished with a polyfunctional material, normally polymeric, having a plurality of functional groups, e.g., hydroxy, amino, mercapto, carboxy, ethylenic, aldehyde, etc., as sites for linking. Alternatively a support may be used. The support can have any of a number of shapes, such as particle including bead, film, membrane, tube, well, strip, rod, and the like. For supports in which photosensitizer is incorporated, the surface of the support is, preferably, hydrophilic or capable of being rendered hydrophilic and the body of the support is, preferably, hydrophobic. The support may be suspendable in the medium in which it is employed. Examples of suspendable supports, by way of illustration and not limitation, are polymeric materials such as latex, lipid bilayers, oil droplets, cells and hydrogels. Other support compositions include glass, metals, polymers, such as nitrocellulose, cellulose acetate, poly(vinyl chloride), polyacrylamide, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials. Attachment of binding agents to the support may be direct or indirect, covalent or non-covalent and can be accomplished by well-known techniques, commonly available in the literature as discussed above. See, for example, “Immobilized Enzymes,” Ichiro Chibata, supra. The surface of the support will usually be polyfunctional or be capable of being polyfunctionalized or be capable of binding to a target-binding moiety, or the like, through covalent or specific or non-specific non-covalent interactions.

The cleavage-inducing moiety may be associated with the support by being covalently or non-covalently attached to the surface of the support or incorporated into the body of the support. Linking to the surface may be accomplished as discussed above. The cleavage-inducing moiety may be incorporated into the body of the support either during or after the preparation of the support. In general, the cleavage-inducing moiety is associated with the support in an amount sufficient to achieve a sufficient amount of active species. Generally, the amount of cleavage-inducing moiety is determined empirically.

### Photosensitizers as Cleavage-Inducing Moieties

As mentioned above, the preferred cleavage-inducing moiety is a photosensitizer that produces singlet oxygen. As used herein, “photosensitizer” refers to a light-adsorbing molecule that when activated by light converts molecular oxygen into singlet oxygen. Photosensitizers may be attached directly or indirectly, via covalent or non-covalent linkages, to the binding agent of a class-specific reagent. Guidance for constructing of such compositions, particularly for antibodies as binding agents, available in the literature, e.g. in the fields of photodynamic therapy, immunodiagnostics, and the like. The following are exemplary references: Ullman, et al., Proc. Natl. Acad. Sci. USA 91, 5426 5430 (1994); Strong et al, Ann. New York Acad. Sci., 745: 297 320 (1994); Yarmush et al, Crit. Rev. Therapeutic Drug Carrier Syst., 10: 197 252 (1993); Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; and the like.

Likewise, there is guidance in the literature regarding the properties and selection of suitable photosensitizers. The following are exemplary references: Wasserman and R. W. Murray. Singlet Oxygen. (Academic Press, New York, 1979); Baumstark, Singlet Oxygen, Vol. 2 (CRC Press Inc., Boca Raton, Fla. 1983); and Turro, Modem Molecular Photochemistry (University Science Books, 1991).

The photosensitizers are sensitizers for generation of singlet oxygen by excitation with light. The photosensitizers include dyes and aromatic compounds, and are usually compounds comprised of covalently bonded atoms, usually with multiple conjugated double or triple bonds. The compounds typically absorb light in the wavelength range of about 200 to about 1,100 nm, usually, about 300 to about 1,000 nm, preferably, about 450 to about 950 nm, with an extinction coefficient at its absorbance maximum greater than about 500 M⁻¹cm⁻¹, preferably, about 5,000 M⁻¹cm⁻¹, more preferably, about 50,000 M⁻¹cm⁻¹, at the excitation wavelength. The lifetime of an excited state produced following absorption of light in the absence of oxygen will usually be at least about 100 nanoseconds, preferably, at least about 1 millisecond. In general, the lifetime should be sufficiently long to permit cleavage of a linkage in a reagent. Such a reagent is normally present at concentrations as discussed below. The photosensitizer excited state usually has a different spin quantum number (S) than its ground state and is usually a triplet (S=1) when the ground state, as is usually the case, is a singlet (S=0). Preferably, the photosensitizer has a high intersystem crossing yield. That is, photoexcitation of a photosensitizer usually produces a triplet state with an efficiency of at least about 10%, desirably at least about 40%, preferably greater than about 80%.

Photosensitizers chosen are relatively photostable and, preferably, do not react efficiently with singlet oxygen. Several structural features are present in most useful photosensitizers. Most photosensitizers have at least one and frequently three or more conjugated double or triple bonds held in a rigid, frequently aromatic structure. They will frequently contain at least one group that accelerates intersystem crossing such as a carbonyl or imine group or a heavy atom selected from rows 3 6 of the periodic table, especially iodine or bromine, or they may have extended aromatic structures.

A large variety of light sources are available to photo-activate photosensitizers to generate singlet oxygen. Both polychromatic and monchromatic sources may be used as long as the source is sufficiently intense to produce enough singlet oxygen in a practical time duration. The length of the irradiation is dependent on the nature of the photosensitizer, the nature of the cleavable linkage, the power of the source of irradiation, and its distance from the sample, and so forth. In general, the period for irradiation may be less than about a microsecond to as long as about 10 minutes, usually in the range of about one millisecond to about 60 seconds. The intensity and length of irradiation should be sufficient to excite at least about 0.1% of the photosensitizer molecules, usually at least about 30% of the photosensitizer molecules and preferably, substantially all of the photosensitizer molecules. Exemplary light sources include, by way of illustration and not limitation, lasers such as, e.g., helium-neon lasers, argon lasers, YAG lasers, He/Cd lasers, and ruby lasers; photodiodes; mercury, sodium and xenon vapor lamps; incandescent lamps such as, e.g., tungsten and tungsten/halogen; flashlamps; and the like.

Examples of photosensitizers that may be utilized include Hypocrellin A, Tetraphenylporphyrin, Hypocrellin B, Halogenated derivatives of rhodamine dyes, Hypericin metallo-Porphyrins, Halogenated derivatives of fluorescein, Phthalocyanines dyes, Rose Bengal, Naphthalocyanines, Merocyanine 540, Texaphyrin-type macrocycles, Methylene blue, Hematophorphyrin, 9-Thioxanthone, 9,10-Dibromoanthracene, Chlorophylls, Benzophenone, Phenaleone, Chlorine 6, Protoporphyrin, Perylene, Benzoporphryin A monacid, Benzoporphryin B monacid, and the like.

In certain embodiments the photosensitizer moiety comprises a support, as discussed above with respect to the cleavage-inducing moiety. The photosensitizer may be associated with the support by being covalently or non-covalently attached to the surface of the support or incorporated into the body of the support as discussed above. In general, the photosensitizer is associated with the support in an amount sufficient to achieve the sufficient amount of singlet oxygen. Generally, the amount of photosensitizer is determined empirically. Photosensitizers used as the photosensitizer are preferably relatively non-polar to assure dissolution into a lipophilic member when the photosensitizer is incorporated in, for example, a latex particle to form photosensitizer beads, e.g. as disclosed by Pease et al., U.S. Pat. No. 5,709,994. For example, the photosensitizer rose bengal is covalently attached to 0.5 micron latex beads by means of chloromethyl groups on the latex to provide an ester linking group, as described in J. Amer. Chem. Soc., 97: 3741 (1975).

In one aspect, a class-specific reagent comprises a first binding agent that is an antibody and a cleavage-inducing moiety that is a photosensitizer, such that the photosensitizer is covalently linked to the antibody, e.g. using well know techniques as disclosed in Strong et al (cited above); Yarmush et al (cited above); or the like. Alternatively, a class-specific reagent comprises a solid phase support, e.g. a bead, to which a photosensitizer is covalently or non-covalently attached and an antibody is attached, preferably convalently, either directly or by way of a functionalized polymer, such as amino-dextran, or the like.

### Methods of Using

In conducting the methods, a combination of the assay components can be made, including the tagged HIV RT subunits, the eTags, and, in some embodiments, the cleaving probe. Generally, assay components may be combined in any order. In certain applications, however, the order of addition may be relevant. For example, one may wish to monitor competitive binding, such as in a quantitative assay. For another example, one may wish to monitor the stability of an assembled complex. In such applications, reactions may be assembled in stages, and may require incubations before the complete mixture has been assembled, or before the cleaving reaction is initiated.

The amounts of each reagent can usually be determined empirically. In general, the amounts of the tagged HIV RT subunits in the heterodimerization assay are provided in equimolar concentrations, but one of the subunits can be at a molar excess of at about 1.5 or more. Where one is determining the effect of a compound on formation of heterodimeric complexes, the compound can be added to the plates prior to, simultaneously with, or after addition of the tagged subunits, depending on the effect being monitored.

The His-tagged RT subunit can be incubated under conditions that provide for binding of the subunit to the surface of a His-binding plate. The His-tagged RT subunit can be in an aqueous medium, generally at a physiological pH maintained by a buffer at a concentration in the range of about 0.1 to 200 mM. Conventional buffers can be used, as well as other conventional additives as useful, such as salts, stabilizers, and the like. The incubation temperatures normally range from about 4° C. to 70° C., usually from about 15° C. to 45° C., more usually 25° C.

After His-tagged RT subunit binds to His-binding plate, the differently-tagged second RT subunit can be added under conditions that provide for binding of the second RT subunit to the first RT subunit bound to the surface of the His-binding plate. The conditions can be an aqueous medium, generally at a physiological pH maintained by a buffer at a concentration in the range of about 0.1 to 200 mM, and incubation temperatures normally range from about 4° C. to 70° C., usually from about 15° C. to 45° C., more usually 25° C.

To the heterodimeric complex thus formed can be added an eTag described above. The eTag preferably can be conjugated to moiety capable of recognizing the tag on the second RT subunit. Thus, for example, if the second RT subunit is FLAG-tagged p66, then an eTag conjugated to anti-FLAG antibody can be used. The conjugated eTag can be at a molar excess of at about 1.5 or more, such as a molar excess of about 2, 3, 5, 10, and the like.

The plates can be washed to remove the unbound RT subunits and eTags. The plate can be treated to cleave the eTags from the complex bound to the plate, releasing the corresponding tag from the complex into solution. The nature of this treatment will depend on the mechanism of action of the cleaving agent. For example, where a photosensitizer is employed as the cleaving agent, activation of cleavage will comprise irradiation of the mixture at the wavelength of light appropriate to the particular sensitizer used. Alternatively, the cleaving agent can be a chemical compound, such as, for example, methylene blue.

Following cleavage, the sample can be analyzed to determine the identity of tags that have been released and to quantify the released tags. Where an assay employing a plurality of tagged probes is employed, separation of the released tags will generally precede their detection. The methods for both separation and detection are determined in the process of designing the tags for the assay. A preferred mode of separation employs electrophoresis, in which the various tags are separated based on known differences in their electrophoretic mobilities.

In the homodimerization assays, only one of the subunits of the HIV RT is used. Where one is determining the effect of a chemical compound on formation of homodimeric complexes, the compound can be added to the reaction mixture prior to, simultaneously with, or after addition of the tagged subunits, depending on the effect being monitored.

One of the RT subunits can be incubated under conditions that provide for dimerization of the subunit. The conditions can be an aqueous medium, generally at a physiological pH maintained by a buffer at a concentration in the range of about 0.1 to 200 mM, and incubation temperatures normally range from about 4° C. to 70° C., usually from about 15° C. to 45° C., more usually 25° C. Then a HIV RT antibody conjugated to a reagent, such as biotin, can be added to bind the antibody portion to the HIV RT subunit. The biotinylated conjugates can then be contacted with a streptavidinated solid phase support thereby immobilizing the homodimer.

To the homodimeric complex thus formed can be added an eTag described above. The eTag preferably can be conjugated to moiety capable of recognizing the RT subunit. Thus, for example, if the subunit is the p66 subunit, then an eTag conjugated to anti-p66 antibody can be used. The conjugated eTag can be at a molar excess of at about 1.5 or more, such as a molar excess of about 2, 3, 5, 10, and the like.

The plates can be washed to remove the unbound RT subunits and eTags. The plate can be treated to activate the cleaving agent to cleave the tags from the tagged probes that are within the effective proximity of the cleaving agent, releasing the corresponding tag from the bound homodimers into solution, releasing the corresponding tag from the complex into solution. The nature of this treatment will depend on the mechanism of action of the cleaving agent. For example, where a photosensitizer is employed as the cleaving agent, activation of cleavage will comprise irradiation of the mixture at the wavelength of light appropriate to the particular sensitizer used.

Following cleavage, the sample is then analyzed to determine the identity of tags that have been released. Where an assay employing a plurality of tagged probes is employed, separation of the released tags will generally precede their detection. The methods for both separation and detection are determined in the process of designing the tags for the assay. A preferred mode of separation employs electrophoresis, in which the various tags are separated based on known differences in their electrophoretic mobilities.

### Separation of Released Molecular Tags

As mentioned above, molecular tags are designed for separation by a separation technique that can distinguish molecular tags based on one or more physical, chemical, and/or optical characteristics (referred to herein as “separation characteristics”). As also mentioned above, separation techniques that may be used include normal phase or reverse phase HPLC, ion exchange HPLC, capillary electrochromatography, mass spectroscopy, gas phase chromatography, and the like. Preferably, the separation technique selected is capable of providing quantitative information as well as qualitative information about the presence or absence of molecular tags (and therefore, corresponding analytes). In one aspect, a liquid phase separation technique is employed so that a solution, e.g. buffer solution, reaction solvent, or the like, containing a mixture of molecular tags is processed to bring about separation of individual kinds of molecular tags. Usually, such separation is accompanied by the differential movement of molecular tags from such a starting mixture along a path until discernable peaks or bands form that correspond to regions of increased concentration of the respective molecular tags. Such a path may be defined by a fluid flow, electric field, magnetic field, or the like. The selection of a particular separation technique depends on several factors including the expense and convenience of using the technique, the resolving power of the technique given the chemical nature of the molecular tags, the number of molecular tags to be separated, the type of detection mode employed, and the like. Preferably, molecular tags are electrophoretically separated to form an electropherogram in which the separated molecular tags are represented by distinct peaks.

Preferably, the separation is by electrophoresis. Methods for electrophoresis of are well known and there is abundant guidance for one of ordinary skill in the art to make design choices for forming and separating particular pluralities of molecular tags. The following are exemplary references on electrophoresis: Krylov et al, Anal. Chem., 72: 111 R-128R (2000); P. D. Grossman and J. C. Colbum, Capillary Electrophoresis: Theory and Practice, Academic Press, Inc., NY (1992); U.S. Pat. Nos. 5,374,527; 5,624,800; 5,552,028; ABI PRISM 377 DNA Sequencer User's Manual, Rev. A, January 1995, Chapter 2 (Applied Biosystems, Foster City, Calif.); and the like. In one aspect, molecular tags are separated by capillary electrophoresis. Design choices within the purview of those of ordinary skill include but are not limited to selection of instrumentation from several commercially available models, selection of operating conditions including separation media type and concentration, pH, desired separation time, temperature, voltage, capillary type and dimensions, detection mode, the number of molecular tags to be separated, and the like.

In one aspect, during or after electrophoretic separation, the molecular tags are detected or identified by recording fluorescence signals and migration times (or migration distances) of the separated compounds, or by constructing a chart of relative fluorescent and order of migration of the molecular tags (e.g., as an electropherogram). To perform such detection, the molecular tags can be illuminated by standard means, e.g. a high intensity mercury vapor lamp, a laser, or the like. Typically, the molecular tags are illuminated by laser light generated by a He—Ne gas laser or a solid-state diode laser. The fluorescence signals can then be detected by a light-sensitive detector, e.g., a photomultipliet tube, a charged-coupled device, or the like. Exemplary electrophoresis detection systems are described elsewhere, e.g., U.S. Pat. Nos. 5,543,026; 5,274,240; 4,879,012; 5,091,652; 6,142,162; or the like. In another aspect, molecular tags may be detected electrochemically detected, e.g. as described in U.S. Pat. No. 6,045,676.

Electrophoretic separation involves the migration and separation of molecules in an electric field based on differences in mobility. Various forms of electrophoretic separation include, by way of example and not limitation, free zone electrophoresis, gel electrophoresis, isoelectric focusing, isotachophoresis, capillary electrochromatography, and micellar electrokinetic chromatography. Capillary electrophoresis involves electroseparation, preferably by electrokinetic flow, including electrophoretic, dielectrophoretic and/or electroosmotic flow, conducted in a tube or channel of from about 1 to about 200 micrometers, usually, from about 10 to about 100 micrometers cross-sectional dimensions. The capillary may be a long independent capillary tube or a channel in a wafer or film comprised of silicon, quartz, glass or plastic.

In capillary electroseparation, an aliquot of the reaction mixture containing the molecular tags is subjected to electroseparation by introducing the aliquot into an electroseparation channel that may be part of, or linked to, a capillary device in which the amplification and other reactions are performed. An electric potential is then applied to the electrically conductive medium contained within the channel to effectuate migration of the components within the combination. Generally, the electric potential applied is sufficient to achieve electroseparation of the desired components according to practices well known in the art. One skilled in the art will be capable of determining the suitable electric potentials for a given set of reagents and/or the nature of the cleaved labels, the nature of the reaction medium and so forth. The parameters for the electroseparation including those for the medium and the electric potential are usually optimized to achieve maximum separation of the desired components. This may be achieved empirically and is well within the purview of the skilled artisan.

Detection may be by any of the known methods associated with the analysis of capillary electrophoresis columns including the methods shown in U.S. Pat. Nos. 5,560,811 (column 11, lines 19 30), 4,675,300, 4,274,240 and 5,324,401, the relevant disclosures of which are incorporated herein by reference. Those skilled in the electrophoresis arts will recognize a wide range of electric potentials or field strengths may be used, for example, fields of 10 to 1000 V/cm are used with about 200 to about 600 V/cm being more typical. The upper voltage limit for commercial systems is about 30 kV, with a capillary length of about 40 to about 60 cm, giving a maximum field of about 600 V/cm. For DNA, typically the capillary is coated to reduce electroosmotic flow, and the injection end of the capillary is maintained at a negative potential.

For ease of detection, the entire apparatus may be fabricated from a plastic material that is optically transparent, which generally allows light of wavelengths ranging from about 180 to about 1500 nm, usually about 220 to about 800 nm, more usually about 450 to about 700 nm, to have low transmission losses. Suitable materials include fused silica, plastics, quartz, glass, and so forth.

In one aspect, molecular tags are separated by electrophoresis in a microfluidics device. Microfluidics devices are described in, for example, U.S. Pat. Nos. 5,750,015; 5,900,130; 6,007,690; and WO 98/45693; WO 99/19717 and WO 99/15876. Conveniently, an aliquot, generally not more than about 5 μl, is transferred to the sample reservoir of a microfluidics device, either directly through electrophoretic or pneumatic injection into an integrated system or by syringe, capillary or the like. The conditions under which the separation is performed are conventional and will vary with the nature of the products.

From the resulting electrophoretic pattern, the molecular tags, and corresponding analytes, can be identified. This is typically done by placing a fluorescence detector near the downstream end of the separation channel, and constructing a electropherogram of the separated molecular tags, first to determine the separation characteristic (in this case, electrophoretic mobility) as above, and secondly, to measure signal intensity, e.g., peak height or peak area, as a measure of the relative amount of tag associated with each probe. Methods for detecting and quantifying levels of a detectable probe are well known. In one preferred method, the molecular tags are fluorescent labeled. A standard fluorescence-emission source is directed against a detection zone in a downstream portion of the separation medium, and fluorescence emission of the zone is measured by a standard light detector. The signal height or area recorded provides a measure of product and substrate concentration in the sample.

With the above detection information, it is now possible to assign each detected molecular tag to a particular probe in the probe set, and to compare the relative levels of each detectable probe, as a measure of its relatively substrate conversion or ligand binding.

All printed patents and publications referred to in this application are hereby incorporated herein in their entirety by this reference. The examples that follow are intended to illustrate, and should not be construed to limit the claims that follow in any way.

### EXAMPLES

### Materials and Methods

### Example 1

### HIV RT Expression Plasmids

The gene for the 51 kDa subunit of HIV-1 RT was cloned into the pBAD HisB prokaryotic expression system (Invitrogen) between the XhoI and HindIII restriction sites, to give pBAD-His p51. This construct allows for the arabinose-inducible expression of the p51 subunit of RT as an N-terminal polyhistidine (6×His) fusion protein following transformation of an appropriate bacterial strain (E. coli).

The gene for the 66 kDa subunit of HIV-1 RT was cloned into the pBAD FLAG prokaryotic expression system (Invitrogen) to give pBAD-FLAG p66 which can be expressed in an appropriately transformed bacterial strain (E. coli).

### Example 2

### Microplate Assay to Monitor p66-p51 RT Dimerization

The assay was performed by first capturing His-tagged p51 on Ni²⁺-NTA microplates, and then adding FLAG-tagged p66 to form the heterodimer. To the heterodimer complex was added a solution of eTag covalently linked to FLAG antibodies resulting His-p51/FLAG-p66 RT heterodimer complexed to an eTag immobilized on the microplates. After washing to remove unbound material, the eTag was cleaved by the addition of methylene blue, and the amount of released eTags was measured and quantitated by electrophoretic separation.

### Example 3

### Drug Screening

The assay of Example 2 was performed in the presence of calmodulin (CAM), [2′,5′-bis-o-(butyldimethylsilyl)-3′-spiro 5′-(4′-amino-1′,2′-oxothiole-2′,2′-dioxide] (TSAO), and RAE family of proteins (RAE). The concentration of the drugs was varied from 0 μM to 100 μM, and the released eTag quantitated. FIG. 3 shows that as the concentration of the drugs is increased, the heterodimerization of p66 and p51 decreases. The IC₅₀ for CAM, TSAO, and RAE was calculated to be 3.7, 6.4, and 11.6 μM, respectively. These values are comparable to the literature vales of IC₅₀ for CAM, TSAO, and RAE was calculated to be 1.9, 6.7, and 11.7 μM, respectively.

While the preferred embodiments of the invention has been illustrated and described, it will be appreciated that various changes can be made therein without departing from the spirit and scope of the invention.

1. A method for measuring heterodimerization of HIV RT, comprising:
immobilizing a recombinant HIV RT subunit selected from a recombinant p51 subunit and a recombinant p66 subunit on a substrate, wherein each recombinant subunit comprises a tag and wherein the p51 subunit and the p66 subunit each comprise different tags;
contacting the immobilized recombinant subunit with the other recombinant subunit thereby forming a heterodimer;
contacting the heterodimer with an eTag covalently linked to an antibody that specifically binds to the heterodimer; and
determining the amount of complex formed by cleaving the bound eTag and measuring the amount of cleaved eTag.

2. The method of claim 1, wherein the recombinant p51 subunit has a wildtype amino acid sequence or a mutant amino acid sequence.

3. The method of claim 1, wherein the recombinant p66 subunit has a wildtype amino acid sequence or a mutant amino acid sequence.

4. The method of claim 1, wherein the tag is selected from the group consisting of Histidine tag, c-myc tag, strep tag, calmodulin binding protein tag, substance P tag, RYIRS tag (SEQ ID NO: 1), Glu-Glu tag, CBD tag, E tag, GFP tag, GST tag, haemagglutinin tag, T7 tag, Tag 100, V5 tag, S tag, Intein/chitin binding domain tag, Xpress tag, thioredoxin tag, VSV tag and FLAG tag.

5. The method of claim 4, wherein the tag is Histidine tag or FLAG tag.

6. The method of claim 5, wherein the recombinant p51 subunit comprises a Histidine tag and the recombinant p66 subunit comprises FLAG tag.

7. The method of claim 6, wherein the immobilizing comprises contacting a Histidine-tagged p51 subunit to a Histidine-tag-binding substrate.

8. The method of claim 1, wherein cleaving the eTag comprises contacting the eTag with a photosensitizer or a chemi-activated sensitizer.

9. A method for measuring heterodimerization of HIV RT, the method comprising:
contacting immobilized His-tagged p51 subunits with a solution comprising FLAG-tagged p66 RT subunits and incubating under conditions that allow formation of p66/p51 RT heterodimers;
contacting the p66/p51 RT heterodimers with eTags each covalently linked to an anti-FLAG antibody; and
determining the amount of p66/p51 RT heterodimers formed by cleaving the bound eTags and measuring the amount of cleaved eTags.

10. The method of claim 9, wherein cleaving the eTags comprises contacting the eTags with a photosensitizer or a chemi-activated sensitizer.

11. A method for identifying compounds capable of modulating HIV RT heterodimerization, the method comprising:
immobilizing His-tagged p51 subunits on a His-binding substrate;
contacting the immobilized His-tagged p51 with a solution of FLAG-tagged p66 RT subunits in the presence or absence of a test compound capable of modulating the heterodimerization of HIV RT and incubating under conditions that allow formation of p66/p51 RT heterodimers;
contacting the p66/p51 RT heterodimers with eTags each covalently linked to an anti-FLAG antibody;
determining the amount of p66/p51 RT heterodimers formed by cleaving the bound eTags and measuring the amount of cleaved eTags; and
comparing the amount of p66/p51 RT heterodimers formed in the presence of the test compound sample to the amount of p66/p51 RT heterodimers formed in a control sample lacking the test compound, whereby a decrease or an increase in p66/p51 RT heterodimer formation in the presence of the test compound is indicative of the ability of the compound to modulate HIV RT heterodimerization.

12. The method of claim 11, wherein cleaving the eTags comprises contacting the eTags with a photosensitizes or a chemi-activated sensitizer.

13. A method for measuring homodimerization of HIV RT, the method comprising:
contacting to homodimers of recombinant p51 subunit or recombinant p66 subunit, wherein the recombinant p51 subunit or recombinant p66 subunit comprises a tag, eTags each covalently linked to an anti-recombinant p51 subunit antibody or an anti-recombinant p66 subunit antibody;
contacting to the homodimers a cleaving probe which binds the recombinant p51 subunit or the recombinant p66 subunit and has a cleavage-inducing moiety with an effective proximity, thereby bringing the eTags within the effective proximity of the cleaving probe releases the molecular tags, and
determining the amount of homodimers formed by measuring the amount of cleaved eTags.

14. The method of claim 13, wherein the recombinant p51 subunit has a wildtype amino acid sequence or a mutant amino acid sequence.

15. The method of claim 13, wherein the recombinant p66 subunit has a wildtype amino acid sequence or a mutant amino acid sequence.

16. The method of claim 13, wherein the tag is selected from the group consisting of Histidine tag, c-myc tag, strep tag, calmodulin binding protein tag, substance P tag, RYIRS tag (SEQ ID NO: 1), Glu-Glu tag, CBD tag, E tag, GFP tag, GST tag, haemagglutinin tag, T7 tag, Tag 100, V5 tag, S tag, Intein/chitin binding domain tag, Xpress tag, thioredoxin tag, VSV tag and FLAG tag.

17. The method of claim 13, further comprising inducing a sensitizer to generate an active species that cleaves the cleavable linkage(s) within the effective proximity.

18. The method of claim 17, wherein the sensitizer is a photosensitizer.

19. The method of claim 18, further comprising illuminating the photosensitizer to generate an active species that cleaves the eTag(s) within the effective proximity.

20. The method of claim 19, wherein the active species is singlet oxygen.

21. The method of claim 13, wherein the cleaving probe comprises biotin.

22. The method of claim 21, further comprising contacting the homodimers to streptavidin immobilized on a solid surface.

23. A method for identifying compounds capable of modulating HIV RT homodimerization, the method comprising:
forming homodimers of recombinant p51 subunit or recombinant p66 subunit, wherein the recombinant p51 subunit or the recombinant p66 subunit comprises a tag, in the presence or absence of a test compound capable of modulating the homodimerization of HIV RT;
contacting the homodimers with eTags each covalently linked to an anti-recombinant p51 subunit antibody or an anti-recombinant p66 subunit antibody;
contacting the homodimers with a cleaving probe which binds the recombinant p51 subunit or the recombinant p66 subunit and comprises a cleavage-inducing moiety with an effective proximity, thereby bringing the eTag within the effective proximity of the cleaving probe;
determining the amount of homodimers formed by cleaving the eTags and measuring the amount of cleaved eTags; and
comparing the amount of homodimers in the presence of the test compound to the amount of homodimers formed in a control sample lacking the test compound, whereby a decrease or an increase in homodimer formation in the presence of the test compound is indicative of the ability of the compound to modulate homodimerization.

24. The method of claim 23, wherein the cleaving probe comprises biotin.

25. The method of claim 24, further comprising contacting the homodimers to streptavidin immobilized on a solid surface.
